(19) United States
(12) Patent Application Publication
(10) Pub. No.: US 2004/0236308 A1
(43) Pub. Date: Nov. 25, 2004

(54) Kinetic isolation pressurization

(75) Inventors: Steve A. Herweck, Nashua, NH (US); Paul Martakos, Pelham, NH (US); Geoffrey Moodie, Nashua, NH (US); Roger Labrecque, Londonderry, NH (US)

Correspondence Address: LAHIVE & COCKFIELD, LLP., 28 STATE STREET, BOSTON, MA, 02109, US

(73) Assignee: ATRIUM MEDICAL CORP., Hudson, NH

(21) Appl. No.: 10/444,863

(22) Filed: May 22, 2003

(51) Int. Cl. A61M 31/00

(52) U.S. Cl. 604/509; 604/103.01

(57) ABSTRACT

A method of delivering a therapeutic agent to a targeted location within a patient efficiently delivers the agent with a reduced systemic effect. The method includes providing a non-perforated delivery device having at least one wall through which a fluid at first fluid pressure can pass through. The non-perforated delivery device is positioned to provide a radial fluid force against the targeted location. The fluid, including at least one therapeutic agent, is supplied to the therapeutic agent delivery device at the first fluid pressure. The fluid passes through the at least one wall of the delivery device to create a semi-confined space external to the delivery device at a second fluid pressure. The delivery device applies the radial fluid force against the semi-confined space and the fluid disposed therein while simultaneously facilitating the fluid passing through the delivery device to maintain the second fluid pressure in the semi-confined space at the targeted location. The fluid contains at least one therapeutic agent that is distributed to the targeted location in a substantially uniform distribution in an amount sufficient to create a therapeutic effect modulatable by the fluid pressure and a dwell time.

### FIELD OF THE INVENTION

The present invention relates to therapeutic agent delivery, and more particularly to a device and/or system for delivering a therapeutic agent, while pressurized, to a targeted location within a patient to maximize the drug distribution and permeation of the tissue atraumatically.

### BACKGROUND OF THE INVENTION

Drug and agent delivery devices are utilized in a wide range of applications including a number of biological applications. Often, such delivery devices take the form of radially expandable devices. For example, inflatable elastomeric balloons have been proposed for treatment of body passages occluded by disease and for maintenance of the proper position of catheter delivered medical devices within such body passages. In addition, drug eluting stents are placed within body lumens with drugs or agents embedded therein for slow release to the body tissue.

Some elastomeric balloons are made to deliver a liquid or gas that includes a drug, to a targeted location. Unfortunately, a substantial amount of the drug or agent that is delivered to the targeted location does not penetrate the tissue sufficiently at the targeted location to result in a therapeutic effect, and is consequently washed away by blood or other fluid that is flowing past the targeted location. This substantially diminishes the effectiveness of the drugs or agents provided through the delivery device, and increases the likelihood of a systemic effect caused by the large quantity of drug or agent washed into the bloodstream. The drugs or agents must be volumetrically increased in anticipation that they will be principally washed away before therapeutically effecting the targeted tissue area. However, because of the systemic effects, the volume of the drugs or agents must not exceed that which can still be considered safe for exposure by systematic dilution and subsequent systematic distribution throughout the patient's body. The drug or agent must be safe enough in its diluted state to be washed away to other parts of the patient's body and not have unwanted therapeutic or otherwise detrimental effects. There is a delicate balance between making the drugs or agents sufficiently concentrated to have therapeutic characteristics at the targeted location, while also being sufficiently diluted to avoid harmful effects after being washed away.

A further drug and agent delivery vehicle conventionally includes drug eluting stents. It is has been determined that the localized concentration of drug permeation into tissue varies with the existing stent delivery vehicles. The drug concentrations at the struts of the stents are relatively higher than drug concentrations at areas between the struts of the stents. This can adversely affect the therapeutic effect of the drug. More specifically, there can be toxic drug concentrations in some areas of the tissue, while there are inadequate concentrations in other areas.

### SUMMARY OF THE INVENTION

There is a need in the art for a method of delivering a therapeutic agent to a targeted location within a patient efficiently delivers the agent with a reduced systemic effect. The present invention is directed toward further solutions to address this need.

In accordance with one embodiment of the present invention, a method of delivering a therapeutic agent to a targeted location within a body cavity includes providing a non-perforated delivery device having at least one wall through which a fluid at first fluid pressure can pass through. The non-perforated delivery device is positioned to provide a radial fluid force against the targeted location. The fluid, including at least one therapeutic agent, is supplied to the therapeutic agent delivery device at the first fluid pressure. The fluid passes through the at least one wall of the delivery device to create a semi-confined space external to the delivery device at a second fluid pressure. The delivery device applies the radial fluid force against the semi-confined space and the fluid disposed therein while simultaneously facilitating the fluid passing through the delivery device to maintain the second fluid pressure in the semi-confined space at the targeted location. The fluid contains at least one therapeutic agent that is distributed to the targeted location in a substantially uniform distribution in an amount sufficient to create a therapeutic effect modulatable by the fluid pressure and a dwell time.

In accordance with aspects of the present invention, the semi-confined space can include a chamber formed by the targeted location and an external wall of the delivery device, and having an orifice along a perimeter of the therapeutic agent delivery device through which the fluid can flow. The orifice can form upon introduction of the fluid, under pressure, external to the delivery device. The first fluid pressure can be greater than the second fluid pressure. The second fluid pressure can be greater than an ambient pressure external to the delivery device and the semi-confined space. The method can further include supplying the fluid to the delivery device using a catheter coupled with the delivery device. The at least one wall can be collapsible and expandable. The delivery device can apply the radial fluid force against the targeted location comprises introducing the fluid to the delivery device at the first fluid pressure to expand the delivery device to an increased effective diameter, resulting in the application of the radial fluid force.

In accordance with further aspects of the present invention, the at least one wall can be fixed in shape. The delivery device applying the radial fluid force against the targeted location can include implanting the delivery device in the body cavity, the delivery device having an effective diameter greater than an effective diameter of the body cavity. The method can further include the radial fluid force expanding the body cavity to between about 101% and about 150% of a pre-implantation body cavity effective diameter. The delivery device can be an irrigating shaped form. The method can further include adjusting the dwell time to modulate an amount of therapeutic agent delivered to the targeted location. At least one of the fluid pressure, a concentration of the therapeutic agent in the fluid, and the dwell time can be modulated to control an amount of therapeutic agent delivered to the targeted location.

In accordance with one embodiment of the present invention, a therapeutic agent delivery device suitable for positioning at a targeted location within a body cavity includes a non-perforated wall structure having a porosity enabling a fluid to pass through at a first fluid pressure, the fluid including at least one therapeutic agent. At least one supply aperture is formed in the wall structure providing access for supplying the fluid to the therapeutic agent delivery device. The wall structure is sized to generate a radial fluid force against the targeted location upon implantation to enable creation of a semi-confined space using the fluid at a second fluid pressure. Further, the wall structure applies the radial fluid force against the targeted location while simultaneously facilitating the fluid passing through the wall structure to maintain the second fluid pressure in the semi-confined space external to the wall structure at the targeted location, such that the therapeutic agent contained within the fluid is substantially uniformly distributed to the targeted location in a substantially in an amount sufficient to create a therapeutic effect modulatable by the fluid pressure and a dwell time.

In accordance with aspects of the present invention, the semi-confined space includes a chamber formed by an the targeted location and an external side of the wall structure, and has an orifice along a perimeter of the therapeutic agent delivery device through which the fluid can flow. The orifice can form upon introduction of the fluid, under pressure, external to the wall structure. The first fluid pressure can be greater than the second fluid pressure. The second fluid pressure can be greater than an ambient pressure external to the therapeutic agent delivery device and the semi-confined space.

Access for supplying the fluid to the therapeutic agent delivery device can include a catheter coupled with the at least one supply aperture. The wall structure can be collapsible and expandable. The radial fluid force against the targeted location results from introduction of the fluid to the therapeutic agent delivery device at the first fluid pressure.

In accordance with further aspects of the present invention, the wall structure can be fixed in shape. The radial fluid force against the targeted location can result from implantation of the therapeutic agent delivery device in the body cavity. The radial fluid force can expand the body cavity to between about 101% and about 150% of a pre-implantation body cavity effective diameter. The wall structure can include an irrigating shaped form.

In accordance with aspects of the present invention, the method can further include adjusting the dwell time to modulate an amount of therapeutic agent delivered to the targeted location. The method can also include modulating at least one of the fluid pressure, a concentration of the therapeutic agent in the fluid, and the dwell time to modulate an amount of therapeutic agent delivered to the targeted location.

### BRIEF DESCRIPTION OF THE DRAWINGS

The present invention will become better understood with reference to the following description and accompanying drawings, wherein:

FIG. 1 is a side elevational view in cross-section of a radially expandable device according to the teachings of the present invention, illustrating the device in a first, reduced diameter configuration;

FIG. 2 is a side elevational view in cross-section of the radially expandable device of FIG. 1, illustrating the device in a second, increased diameter configuration;

FIG. 3 is a schematic representation of the microstructure of a section of the wall of an expanded fluoropolymer irrigating shaped form used during the manufacturing process of the present invention to yield the radially expandable device of the present invention;

FIG. 4 is diagrammatic illustration of a therapeutic drug delivery system according to one aspect of the present invention;

FIGS. 5A, 5B, and 5C are cross-sectional illustrations of the expandable device at the internal wall of a body lumen, according to one aspect of the present invention;

FIGS. 6A, 6B, and 6C are perspective illustrations of stents for use in conjunction with the present invention;

FIG. 7 is a flow chart illustrating an example method of applying a therapeutic drug according to one aspect of the present invention;

FIG. 8 is a flow chart illustrating an example method of forming a polymeric body, according to one aspect of the present invention; and

FIG. 9 is a flow chart illustrating example embodiment of applying a therapeutic gas to a targeted location within a patient's body.

### DETAILED DESCRIPTION

An illustrative embodiment of the present invention relates to a device, system, and method for delivering a therapeutic agent or drug to a targeted location within a patient's bodyto maximize drug delivery and permeation of body tissue by the drug or agent in an atraumatic manner. The present invention delivers the therapeutic agent or drug, both extra-cellularly and intra-cellularly, relying on a kinetic isolation pressurization effect (hereinafter “KIP effect”).

The phrase “therapeutic drug and/or agent” and variations thereof are utilized interchangeably herein to indicate single or multiple therapeutic drugs, single or multiple therapeutic agents, or any combination of single or multiple drugs or agents. As such, any subtle variations of the above phrase should not be interpreted to indicate a different meaning, or to refer to a different combination of drugs or agents. The present invention is directed toward the delivery of therapeutic drugs and/or agents, or any combination thereof, as understood by one of ordinary skill in the art.

The KIP effect can be defined as the resulting effect of applying a pressurized fluid to an isolated or targeted location to create and maintain a semi-confined space (the isolated or targeted location forming at least one portion of the semi-confined space) to improve permeability by, and deposition of, a therapeutic drug or agent into the isolated or targeted location of body tissue.

More specifically, the KIP effect makes use of a flowing fluid directed under pressure at a targeted location requiring the treatment offered by the particular drug or agent being delivered. The pressure of the fluid as it makes atraumatic contact with the targeted location creates a region of fluid containing a substantially uniform distribution and concentration of one or more therapeutic agents. The region of fluid enables a uniform application or deposition of the therapeutic agent(s) for a desired dwell time or residence time, which results in improved tissue permeation by the therapeutic drug(s) or agent(s). The more uniform deposition of the therapeutic drug(s) or agent(s) and the improved tissue permeation by the therapeutic drug(s) or agent(s) results in a more even concentration of the therapeutic drug(s) or agents(s) in the tissue being treated.

As such, the strength or concentration of the drug or agent contained within the fluid can be maintained or increased while the overall dosemetric or volumetric amount of the drug or agent is reduced relative to the known oral and systemic drug delivery methods discussed previously, while still resulting in a therapeutic effect. Any excess volume of drug or agent that does not permeate the tissue of the targeted location is diluted and washes away with the pressurized fluid. However, the fluid containing the drug or agent can be substantially more concentrated in terms of drug or agent content than with other known methods. Because the therapeutic drug or agent becomes quickly diluted after exiting the targeted location, and because there is a lower overall dosemetric amount of agent or drug relative to other known methods, the likelihood of causing an unwanted therapeutic or otherwise detrimental effect on other parts of the patient's body is reduced. In addition, the increased permeability of the tissue by the drug or agent results in the targeted location receiving an increased amount of the drug or agent, relative to prior methods, for more effective treatment.

In short, the fluid applied to the targeted location can be more concentrated with the therapeutic drug or agent, but in less overall dosemetric quantity, than with prior methods because the isolation and pressurization of the KIP effect substantially improves the permeation of the tissue by the drug or agent. The improved permeation requires less dosemetric amounts of the therapeutic drug or agent, to result in an improved therapeutic effect relative to known oral and systemic distribution methods.

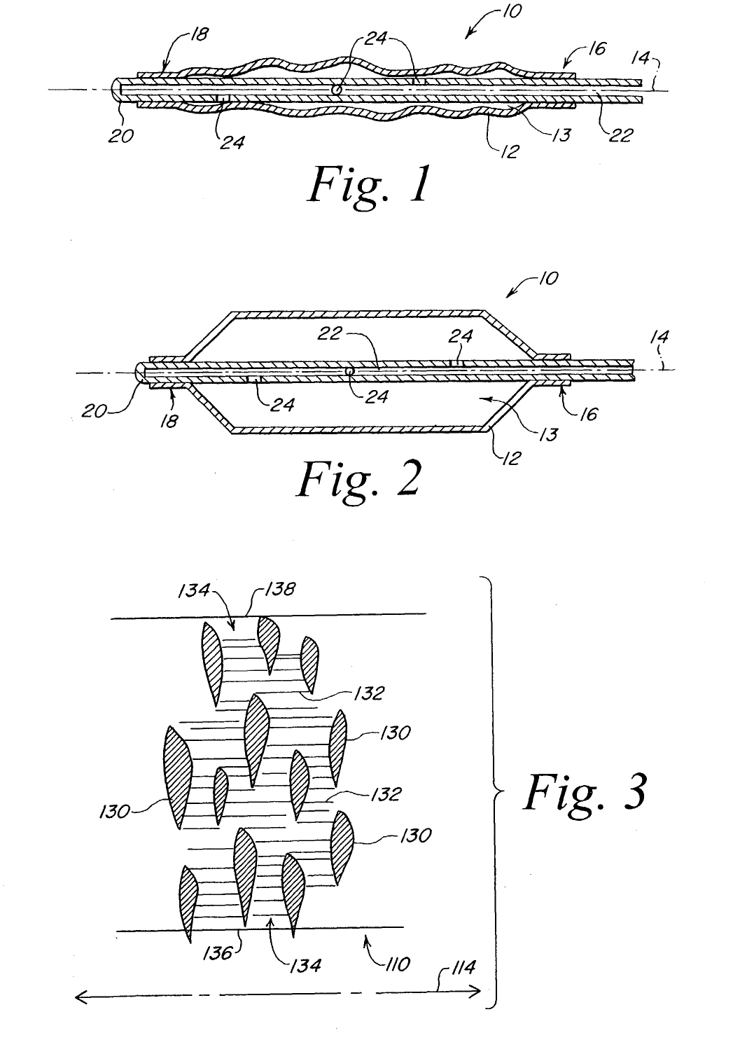

FIGS. 1 through 9, wherein like parts are designated by like reference numerals throughout, illustrate example embodiments of devices, systems, and methods for forming and delivering fluids to a patient utilizing the KIP effect, according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

In accordance with one example embodiment of the present invention, a radially expandable device 10 having an irrigating shaped form, such as body 12 constructed of a generally inelastic, expanded fluoropolymer material, is illustrated in FIGS. I and 2. Expandable devices provided by the present invention are suitable for a wide range of applications including, for example, a range of medical treatment applications. Exemplary biological applications include use as a catheter balloon for treatment of implanted vascular grafts, stents, prosthesises, or other type of medical implant, and treatment of any body cavity, space, or hollow organ passage(s) such as blood vessels, the urinary tract, the intestinal tract, nasal cavity, neural sheath, bone cavity, kidney ducts, etc. The catheter balloon can be of the type with a catheter passing through a full length of the balloon, or of the type with a balloon placed at an end of a catheter. Additional examples include as a device for the removal of obstructions such as emboli and thrombi from blood vessels, as a dilation device to restore patency to an occluded body passage as an occlusion device to selectively deliver a means to obstruct or fill a passage or space, and as a centering mechanism for transluminal instruments and catheters. The expandable device 10 can also be used as a sheath for covering conventional catheter balloons to control the expansion of the conventional balloon.

The body 12 of the example radially expandable device 10 is deployable upon application of an expansion force from a first, reduced diameter configuration, illustrated in FIG. 1, to a second, increased diameter configuration, illustrated in FIG. 2. The body 12 of the radially expandable device 10 preferably features a monolithic construction, i.e., the body 12 is a singular, unitary article of generally homogeneous material. The example body 12 is manufactured using an extrusion and expansion process described in detail in U.S. patent application No. 10/131396, filed Apr. 22, 2002, which is hereby incorporated herein by reference. Alternative methods can include use of plasma treated PTFE, and PTFE stretched with additional wetting as described in U.S. patent application No. 09/678,765 filed Oct. 3, 2000, hereby incorporated by reference. In addition, the radially expandable device 10 is merely one example embodiment. Any therapeutic drug or agent delivery device capable of sustaining a desired elevated pressure as described below and delivering the fluid with therapeutic drug or agent under pressure to an isolated location, as understood by one of ordinary skill in the art, can be utilized in practicing the KIP effect. As shown, the expandable member 10 is an expandable irrigating shaped form that can be coupled with a catheter or other structure able to provide fluid (in the form of a slurry of nanoparticles, semi-solid, solid, gel, liquid or gas) to the irrigating shaped form under pressure.

The example process yields a body 12 characterized by a non-perforated seamless construction of inelastic, expanded fluoropolymer. The fluoropolymer has a predefined size and shape in the second, increased diameter configuration. The body 12 can be dependably and predictably expanded to the predefined, fixed maximum diameter and to the predefined shape independent of the expansion force used to expand the device.

Alternatively, it should be noted that the aforementioned methods of manufacture relate to the creation of an elastomeric irrigating shaped form suitable for illustrative purposes as an example therapeutic delivery device. The radially expandable device 10 can be made of a number of other different materials as well, as understood by one of ordinary skill in the art. For example, suitable fluoropolymer materials include polytetrafluoroethylene (“PTFE”) or copolymers of tetrafluoroethylene with other monomers may be used. Such monomers include ethylene, chlorotrifluoroethylene, perfluoroalkoxytetrafluoroethylene, or fluorinated propylenes such as hexafluoropropylene. PTFE is utilized most often. Accordingly, while the radially expandable device 10 can be manufactured from various fluoropolymer materials, and the manufacturing methods of the present invention can utilize various fluoropolymer materials, the description set forth herein refers specifically to PTFE.

Referring specifically to FIG. 2, the body 12 of the radially expandable device 10 is preferably generally tubular in shape when expanded, although other cross-sections, such as rectangular, oval, elliptical, or polygonal, can be utilized. The cross-section of the body 12 is preferably continuous and uniform along the length of the body. However, in alternative embodiments, the cross-section can vary in size and/or shape along the length of the body. FIG. 1 illustrates the body 12 relaxed in the first, reduced diameter configuration. The body 12 has a central lumen 13 extending along a longitudinal axis 14 between a first end 16 and second end 18.

A deployment mechanism in the form of an elongated hollow tube 20 is shown positioned within the central lumen 13 to provide a radial deployment or expansion force to the body 12. The radial deployment force effects radial expansion of the body 12 from the first configuration to the second increased diameter configuration illustrated in FIG. 2. The first end 16 and the second end 18 are connected in sealing relationship to the outer surface of the hollow tube 20. The first and second ends 16 and 18 can be thermally bonded, bonded by means of an adhesive, or attached by other means suitable for inhibiting fluid leakage from the first and second ends 16 and 18 between the walls of the body 12 and the tube 20.

The hollow tube 20 includes an internal, longitudinal extending lumen 22 and a number of side-holes 24 that provide for fluid communication between the exterior of the tube 20 and the lumen 22. The tube 20 can be coupled to a fluid source or sources (as later described) to selectively provide fluid to the lumen 13 of the body 12 through the lumen 22 and side-holes 24. The pressure from the fluid provides a radially expandable force on the body 12 to radially expand the body 12 to the second, increased diameter configuration. Because the body 12 is constructed from an inelastic material, uncoupling the tube 20 from the fluid source or otherwise substantially reducing the fluid pressure within the lumen 13 of the body 12, does not generally result in the body 12 returning to the first, reduced diameter configuration. However, the body 12 will collapse under its own weight to a reduced diameter. Application of negative pressure, from, for example, a vacuum source, can be used to completely deflate the body 12 to the initial reduced diameter configuration.

One skilled in the art will appreciate that the radially expandable device 10 is not limited to use with deployment mechanisms employing a fluid deployment force, such as hollow tube 20. Other known deployment mechanisms can be used to radially deploy the radially expandable device 10 including, for example, mechanical operated expansion elements, such as mechanically activated members or mechanical elements constructed from temperature activated materials such as nitinol.

Various fluoropolymer materials are suitable for use in the present invention. Suitable fluoropolymer materials include, for example, polytetrafluoroethylene (“PTFE”) or copolymers of tetrafluoroethylene with other monomers may be used. Such monomers include ethylene, chlorotrifluoroethylene, perfluoroalkoxytetrafluoroethylene, or fluorinated propylenes such as hexafluoropropylene. PTFE is utilized most often. Accordingly, while the radially expandable device 10 can be manufactured from various fluoropolymer materials, and the manufacturing methods of the present invention can utilize various fluoropolymer materials, the description set forth herein refers specifically to PTFE.

FIG. 3 is a schematic representation of the microstructure of the walls of an ePTFE irrigating shaped form 110, such as the body 12, as formed by an extrusion and expansion process. For purposes of description, the microstructure of the irrigating shaped form 110 has been exaggerated. Accordingly, while the dimensions of the microstructure are enlarged, the general character of the illustrated microstructure is representative of the microstructure prevailing within the irrigating shaped form 110.

The microstructure of the ePTFE irrigating shaped form 110 is characterized by nodes 130 interconnected by fibrils 132. The nodes 130 are generally oriented perpendicular to the longitudinal axis 114 of the irrigating shaped form 110. This microstructure of nodes 130 interconnected by fibrils 132 provides a microporous structure having microfibrillar spaces that define through-pores or channels 134 extending entirely from the inner wall 136 and the outer wall 138 of the irrigating shaped form 110. The through-pores 134 are perpendicularly oriented (relative to the longitudinal axis 114), intemodal spaces that traverse from the inner wall 136 to the outer wall 138. The size and geometry of the through- pores 134 can be altered through the extrusion and stretching process, as described in detail in Applicants' U.S. patent application Ser. No. 09/411797, filed on Oct. 1, 1999, which is incorporated herein by reference, to yield a microstructure that is impermeable, semi-impermeable, or permeable. However, it should be noted that the invention is not limited to this method of manufacture. Rather, the application referred to is merely one example method of producing an expandable device.

The size and geometry of the through-pores 134 can be altered to form different orientations. For example, by twisting or rotating the ePTFE irrigating shaped form 110 during the extrusion and/or stretching process, the micro-channels can be oriented at an angle to an axis perpendicular to the longitudinal axis 114 of the irrigating shaped form 110. The expandable device 10 results from the process of extrusion, followed by stretching of the polymer, and sintering of the polymer to lock-in the stretched structure of through-pores 134.

The microporous structure of the through pores 134 of the material forming the expandable device 10 enable permeation of the wall of the expandable device 10 without the need for creating perforations in the expandable device 10. The microporous structure of the device enables a more controllable, and more even, distribution of fluid through the walls of the expandable device 10 relative to a perforated device with fluid exiting the device only at the perforations. Thus, the non-perforated structure of the expandable device 10 contributes to the effective distribution of the fluid by the expandable device 10 as described herein. Some known methods for distribution of a fluid in a body lumen include the use of a perforated balloon. The fluid emits through the perforations into the body lumen. The non-perforated microporous structure of the through pores 134 of the present invention provides a far greater percentage of surface area through which the fluid can flow relative to specific perforations. The far greater plurality of locations (i.e., through pores 134) through which the fluid permeates the expandable device 10 relative to specific perforations made in a wall enables a more even and complete distribution of fluid to the targeted location, and a more even distribution of fluid pressure to better execute the KIP effect.

In accordance with one embodiment, the ePTFE irrigating shaped form 110, and the resultant expandable device 10, has a fine nodal structure that is uniform throughout the cross section and length of the ePTFE irrigating shaped form. The uniform fine nodal structure provides the expandable device 10 with improved expansion characteristics as the expandable device dependably and predictably expands to the second diameter. The fine nodal structure can be characterized by nodes having a size and mass less than the nodes found in conventional ePTFE grafts, for example in the range of 25 μm−30 μm. Additionally, the spacing between the nodes, referred to as the intemodal distance, and the spacing between the fibers, referred to as the interfibril distance, can also be less than found in conventional ePTFE grafts, for example in the range of 1 μm−5 μm. Moreover, the intemodal distance and the interfibril distance in the example embodiment can be uniform throughout the length and the cross section of the ePTFE irrigating shaped form. The uniform nodal structure can be created by forming the billet with a uniform lubricant level throughout its cross section and length. Stretching the tubular extrudate at higher stretch rates, for example at rates greater than 1 in/s, yields the fine nodal structure. Preferably, the extrudate is stretched at a rate of approximately 10 in/s or greater. The nodal structure can also be non-uniform, by varying the location and amount of lubrication and stretching processes.

In the instance of the fluid inflating the body 12 of the radially expandable device 10, the fluid can pass through the body 12 in a pressurized weeping manner, and be applied to a target location in the patient body, as discussed further below. The fluid, in such an instance, can contain one or more drugs having therapeutic properties for healing the affected target location. Example therapeutic drugs and therapeutic agents can include, but are not limited to, those listed in Table 1 below.

TABLE #1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Antiinflammatory Agents | Glucocorticoids, NSAIDS, ibuprofen, acetaminophen, |
| | hydrocortizone acetate, hydrocortizone sodium phosphate |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | microphenonol acid, rapamycin, everolimus, tacrolimus, |
| | paclitaxel, estradiol, dexamethasone, methatrexate, |
| | cilastozol, prednisone, cyclosporine, doxorubicin, |
| | ranpirnas, troglitzon, valsarten, pemirolast |
| Tissue growth stimulants | Bone morphogeneic protein, fibroblast growth factor |
| Gasses | Nitric oxide, super oxygenated O2 |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | SNAP |
| Gases | Nitric oxide, super oxygenated O₂ compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Chemotherapeutic Agents | Cyclosporine, doxorubicin, paclitaxel, tacrolimus, |
| | sirolimus, fludarabine, ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |

Surgical adhesives, anti-adhesion gels and/or films, and tissue-absorbing biological coatings can also be utilized with the present invention and with or without the therapeutic drugs and agents of Table 1. The adhesive-type polymers can include both one and two-part adhesives for use with or without the therapeutic drugs or agents. Examples of the adhesive-type polymers include 2-octyl cyanoacrylate, a patient's own plasma mixed with a suspension of human derived collagen and thrombin to form a natural biological sealant, fibrin glue derived from preparation of the patient's blood, polymeric hydrogels, and the like. The tissue-absorbing therapeutic agents, as shown in Table 1, can be incorporated into the fluid such as those which include fish oil omega 3 fatty acids, vegetable oils containing fish oil omega 3 fatty acids, other oils or substances suitable for enhancing tissue absorption, adhesion, lipophillic permeation, and any combination thereof. Anti-adhesion film forming gels, solutions, or compounds can be used with or without therapeutic drugs to enhance tissue adhesion of the agents and improve intra-cellular and extra-cellular therapeutic agent permeation simultaneous to reducing traumatic tissue adhesion formation in and around the targeted treatment site. Reduced tissue adhesion formation in selected areas prone to adhesion formation, such as stented vessels, dilated urethras, and the like, benefit from such an anti-adhesion therapeutic delivery method.

The intemodal distance and the interfibral distance can be varied to control over a relatively larger range, to allow a fluid to pass through the through-pores or channels 134. The size of the through-pores or channels 134 can be selected through the manufacturing process, for example as described in detail in U.S. patent application Ser. No. 09/411797, previously incorporated herein by reference. The internodal distance of microstructure of the wall within the microporous region, and hence the width of the through-pores or channels 134, can be approximately 1 μm to approximately 150 μm. Internodal distances of this magnitude can yield flow rates of approximately 0.01 μl/min to approximately 100 ml/min of fluid through the wall of the body 12.

The internodal distances can also vary at different locations along the microporous structure to result in the channels 134 being of different sizes in different locations or regions. This enables different flow rates to occur through different areas of the same microporous structure at a substantially same fluid pressure.

The different flow rates achieved by the radially expandable device 10 can contribute to variations in fluid pressure during inflation of the expandable device 10, and also enable a variation in dwell time of the expandable device 10 at a targeted location requiring therapeutic treatment. An additional factor can include the relative viscosity of the fluid(s) to each other for mixing purposes, and the resulting fluid viscosity of the therapeutic agent. The more viscous, the more resistant to flow, thus the longer dwell time required to apply a sufficient amount of agent.

Dwell time is a measurement of the amount of time the expandable device 10 is disposed within the patient body applying one or more therapeutic agents to a location within the patient body, such as a targeted location. The targeted location is a location requiring therapeutic treatment. The ability to vary the size and shape of the through-pores or channels 134 enables modification of the dwell time. If a longer dwell time is desired, the size and shape of the through-pores 134 can be varied to allow less fluid to pass through. Likewise, if a shorter dwell time is desired with the same amount of therapeutic fluid to be applied, the through-pores 134 can be varied to allow more fluid to pass through at a faster rate. In addition, the dwell time can be affected by the pressurization of the fluid being absorbed by the tissue of the body lumen or cavity in accordance with one example embodiment of the present invention and later described herein.

The microporous structure of the through-pores 134 is such that the fluid pressure of the fluid passing through can vary over a substantial range and still result in substantially the same rate of fluid flow through the through-pores 134. For example, for a predetermined range of fluid pressures, the rate of fluid flow through the through-pores 134 remains substantially constant for a given embodiment. Alternatively, the percentage of change of the rate of fluid flow can be made less than a given percentage of change of fluid pressure. The pressure within the expandable device 10 can range, for example in one embodiment involving the pressurization of the fluid external to the expandable device 10, up to about six atmospheres. Other ranges that have been shown to work with the expandable device 10 include pressures in the range of two atmospheres to four atmospheres. One result of having relatively lower fluid pressure within the flexible expandable device 10 is that the expandable device 10 is able to conform to the shape of the body lumen or cavity within which the expandable device 10 operates, rather than the expandable device 10 causing trauma to the body tissue from over-expansion.

The pressure within the expandable device 10 can be supplied in a constant, variable, or intermittent amount by varying the flow of fluid to the expandable device 10. The variation of fluid pressure inside the expandable device 10 can influence a variation of the fluid pressure external to the expandable device 10 as described further below.

Some of the pressure internal to the expandable device 10 translates to fluid pressure external to the expandable device 10. The pressurized fluid exits the expandable device 10 and permeates the tissue of the targeted location as described further below.

In accordance with one example embodiment, FIG. 4 illustrates a therapeutic drug delivery system 200. The expandable device 10 is in fluid communication with a first storage container 212 through a tubular coupling 214. The example expandable device 10 is also in fluid communication with a second storage container 216 through a second tubular coupling 218. Different amounts of a component or components in fluid form from the first storage container 212 and the second storage container 216 can be mixed together within the expandable device 10 prior to exit from the expandable device 10 and entry into the patient. In addition, the coupling with the expandable device 10 is removable to switch connections to storage containers easily.

There can be a number of additional storage containers represented by storage container 222 with tubular coupling 224 and storage container 226 with tubular coupling 228. Each storage container 212, 216, 222, and 226 can maintain a separate component until mixing occurs. Therefore, the number of storage containers can vary. In addition, the type of storage container can vary. Any of the storage containers 212, 216, 222, and 226 can be suitable for holding a solid, liquid, or gas. More specifically, the first storage container 212 can be designed to hold a liquid, while the second storage container 216 can be designed to hold a gas, or vice versa, or one or the other could hold another of the solids, liquids, or gases. It is not necessary for any single container design to be able to hold solids, liquids, and/or gases, but such a design would be functional with the present invention.

Alternatively, different designs can be provided depending on the physical state of the component being stored. The solid that can be held by the storage containers 212, 216, 222, and 226 can be in powder form, such that the solid can be easily transferred to the expandable device 10 for mixing with a liquid or gas. Further, the storage containers 212, 216, 222, and 226 can be heated or cooled to maintain a desired temperature of the component being stored, if necessary.

It should be appreciated that any number of storage containers required for a specific embodiment, from one to a plurality, is considered to be anticipated by the present description and illustrations.

A controller 220 can be included along the first tubular coupling 214 to vary or control the amount of component fluid passing through to the expandable device 10. The controller 220 can take a number of different forms. Primarily, the controller 220 restricts flow and/or diverts flow from the first storage container 212, and any additional containers. The controller 220 can include a simple valve with adjustable flow rates, or can be more elaborate as understood by one of ordinary skill in the art. The example controller can also introduce sufficient pumping action to pressurize the fluid supplied by the first storage container 212. Alternatively, the storage container 212 itself can be pressurized. An example controller is a pressure infusor conventionally employed for angioplasty balloon catheter inflation with a pressure gauge. One ore more pressure infusor devices connected to a manifold provides multiple therapeutic element infusion into the device.

In an alternative arrangement, the first tubular coupling 214 can feed to the expandable device 10 without the interjection of the controller 220. The amounts of the fluids necessary for the targeted location can be determined by the amount of dilution (or lack thereof) for each fluid separately.

Whether there are multiple components in the storage containers, or single components, and whether the components are in solid, liquid, or gas form, various characteristics of the components can be changed. For example, the components can be diluted or strengthened, heated or cooled, mixed or layered, and the like. In addition, the components can be varied in terms of their supply, e.g., constant, variable, or intermittent flow rates can be provided to the expandable device 10 and through the expandable device 10. Further, the components can be varied in terms of state, e.g., solid powder, semi-solid, nanoparticles, gel, liquid, gaseous, highly viscous liquid, cured coating, intermixed with a polymer such as PTFE, and the like.

In accordance with further embodiments of the present invention, the one or more components can be combined to form a polymeric body with or without a therapeutic agent. For example, the storage container 212 can contain components that create a polymer material. Upon delivery of the components to the expandable device 10, the components cure to form the polymeric structure. Such a structure can be used to seal internal hemorrhages, cover a set of stitches to create a smooth surface, bond body tissues together, coat a diseased or damaged tissue with a protective coating, and the like.

It should be noted that the resulting agent, whether therapeutic or non-therapeutic, can have the physical form including a gas, liquid, powder, gel, micro-particle, and nano-particle.

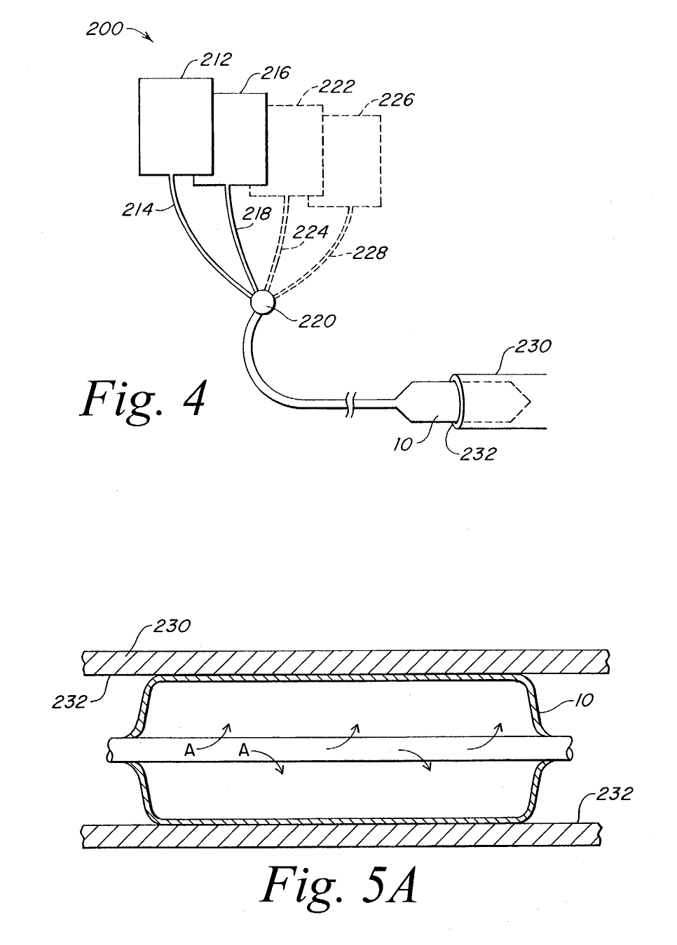

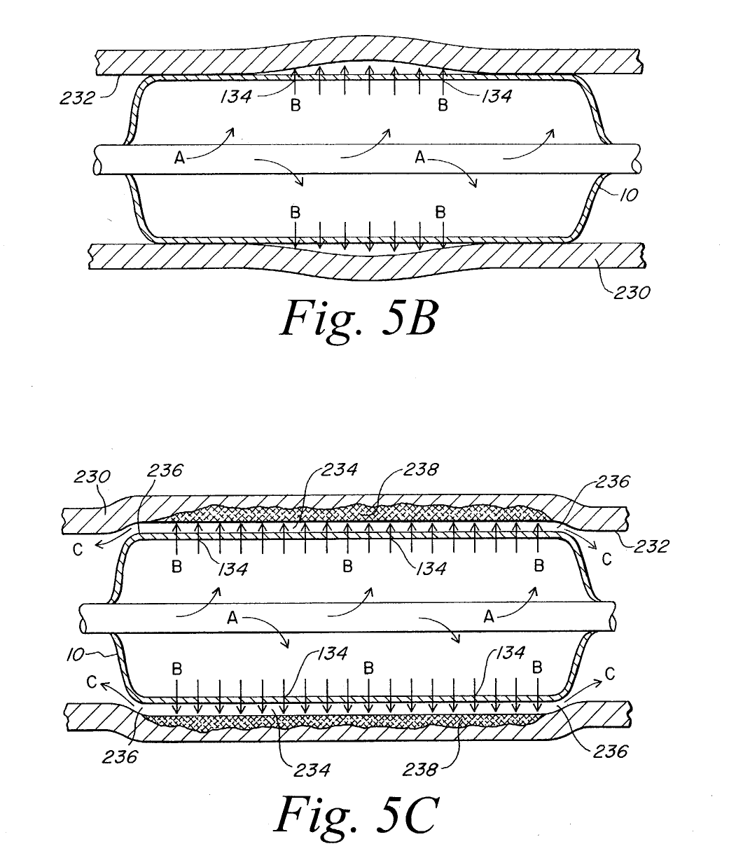

The expandable device 10 is shown inserted into a partial sectional representation of a body cavity or lumen 230 having an internal wall 232 in FIG. 5A. The body cavity or lumen 230 is a small confined hollow space within a patient's body against which pressure can be applied with an expanding device or a device sized slightly larger than the cavity or lumen. Such a space is herein referred to as the body lumen. The body lumen 230 can be, for example, a blood vessel, capillary, or other enclosed structure into which the expandable device 10 can be inserted. Application of the expandable device 10 is discussed further below.

In operation, the expandable device 10 is inserted into the patients body and maneuvered to the targeted location, for example, in the body lumen 230 shown in FIG. 4. The pressure within the expandable device 10 can range over a number of different pressures as understood by one of ordinary skill in the art. For example, the pressure can range up to about six atmospheres in one example embodiment, between about two atmospheres and about four atmospheres according to another example, or another desired range of pressure. The expandable device 10 can inflate, under pressure from an ingressing fluid or agent, to push against the internal wall 232 of the body lumen 230 in which the expandable device 10 is implanted. It should again be noted that the blood vessel representing the body lumen 230 is merely an illustrative example of an appropriate targeted location for introduction of therapeutic agents by the expandable device 10 in accordance with the present invention.

The expandable device 10 is provided in a number of different size ranges, such that the size of the expandable device 10 in fully expanded state is greater than 100% of the inner diameter size of the body lumen or cavity in which the expandable device 10 is placed. In other words, the expandable device 10 inflates and takes up sufficient space within the body lumen or cavity to create a pressure applied by the expandable device 10 against the tissue of the body lumen or cavity. If the expandable device 10 is too small, when it is fully expanded it will not reach the walls of the body lumen, and therefore no contact will be established to generate the KIP effect. If the expandable device 10 is too large, full expansion of the device 10 will cause trauma and possible dissection to the body lumen or cavity. In some instances, this may be desirable (if the desire is to force the healing repair of a vessel, for example). However, in other instances, an expandable device 10 too large for the body lumen or cavity is undesirable. Therefore, the user must select a size appropriate for the task at hand. For example, for the situation where the user requires that the expandable device 10 apply a non-traumatic pressure to the body lumen or cavity, the expandable device 10 can be selected to expand to about 101% to 105%, or up to about 110%, or even 150% of the effective inner diameter of the body lumen or cavity. The effective diameter is essentially an approximation of overall size, which is equivalent to the actual diameter of a circular cross-section, and is equivalent to a diameter-type dimension of a non-circular cross-section. Other size ranges are possible, based on pressure applied to the expandable device 10, strength of the body lumen or cavity, and desire for non-traumatic or traumatic results, as understood by one of ordinary skill in the art.

The characteristics of the expandable device 10 are such that the pressure placed by the expandable device 10 on the internal wall 232 would otherwise hold the expandable device 10 against the internal wall 232 if not for the creation of a semi-confined space 234 in accordance with one example embodiment of the present invention as illustrated in FIG. 5C. The semi-confined space 234 is the area between the expandable device 10 as the expandable device 10 is pressed against the internal wall 232 of the body lumen 230 and a pressurized fluid is forced out of the expandable device 10. The semi-confined space 234 is bordered on one side by the expandable device 10, on an opposite side by the internal wall 232 of the body lumen, and on a third side by a small orifice 236 that forms around the edges of the expandable device 10 where the expandable device ends as the pressurized fluid occupies the space.

To further elaborate, FIG. 5A shows the expandable device 10 inflated via the fluid flowing in the direction of arrows A and pressed against the internal wall 232 of the body lumen 230. In the illustrated state, there is no semi-confined space 234 because the fluid that is expanding the expandable device 10 has not yet passed through the walls of the expandable device 10. Once sufficient fluid has passed through the walls of the expandable device, the fluid remains pressurized and pushes against the internal wall 232 and the outside wall of the expandable device 10 to form the semi-confined space 234. Through compression of the expandable device 10 and the internal wall 232, the semi-confined space 234 is created. FIG. 5B illustrates some fluid gathering external to the expandable device 10 and beginning to form the semi-confined space 234 (however, the space has not been completed as shown). Additional pressurized fluid provided external to the expandable device 10 expands the space to form the semi-confined space 234 as shown in FIG. 5C. Once complete, the semi-confined space 234 reaches the end of the expandable device 10 and the small orifice 236 is created. With additional pressurized fluid provided to the expandable device 10, the pressure external to the expandable device 10 is maintained, the semi-confined space 234 is maintained, and the small orifice 236 remains open. If the pressure of the fluid external to the expandable device falls substantially, then the small orifice 236 will close.

The semi-confined space 234 channels the pressurized fluid emitting through the through-pores 134 of the expandable device 10 in the direction of the arrows B shown. This arrangement causes the therapeutic agents and/or drugs concentrated in the fluid to have complete exposure to the targeted location of the internal wall 232. As such, at least some of the therapeutic agents and/or drugs permeate into the localized cellular space and tissue of the internal wall 232 into a permeation region 238. In addition, some of the fluid creates and then leaks out through the small orifice 236 around the edges of the expandable device 10 in the direction of arrows C. Thus, some of the pressure from within the expandable device 10 carries through to the semi-confined space 234, resulting in the fluid being pressurized against the internal wall 232 of the body lumen 230. Once the fluid exits the semi-confined space 234, the drugs and/or agents contained within the fluid are diluted and subsequently washed away.

The KIP effect is instrumental in creating the semi-confined space 234 between the expandable device 10 and the internal wall 232 of the body lumen 230, and thus creating a more even distribution or deposition of therapeutic drug or agent at the permeation region 238 of the internal wall 232. This semi-confined space 234 is continuously filled with fluid passing through the wall of the expandable device 10 and feeding into the semi-confined space 234. With the continuous fluid movement, and the elevated pressure within the semi-confined space 234, the actual structure of the expandable device 10 does not maintain contact with the internal wall 232 or the permeation region 238 for any extended period. Therefore, a continually churning volume of fluid containing a concentration of at least one therapeutic agent or drug is deposited at the internal wall 232. There is no opportunity for some areas of therapeutic drug or agent to become stagnated in a location on the tissue of the internal wall 232 because the fluid movement constantly churns the therapeutic drug or agent, continually providing a fresh supply and even or substantially uniform deposition.

The continuous churning and re-supply of the fluid containing the at least one therapeutic drug or agent provides a regulated, substantially uniform, therapeutic drug or agent concentration at the tissue. The pressurized fluid also provides for atraumatic delivery or deposition of the therapeutic drugs or agents. Further, there is no structural impediment to drug deposition, such as struts from a stent, or areas of compression by a balloon against the internal wall 232, that may cause pooling of the fluid and thus the therapeutic drug or agent. With an even deposition of a substantially uniform concentration of therapeutic agent or drug, there is an increased efficiency in tissue permeation, and a more even concentration of therapeutic drug or agent permeating the internal wall 232 of the body lumen 230.

The delivery of a therapeutic agent or drug must achieve sufficient concentration at the targeted location for efficacy. Prior methods required use of a substantially higher dosemetric or volumetric amount of drug or agent to attempt to achieve a therapeutic effect at the targeted location relative to the present invention. Prior methods had to include sufficient amounts of a drug or agent to permeate the tissue while also working around structures such as stent struts, and while being washed away from the targeted location. Alternatively, prior methods supplied a substantially greater amount of drug to a patient using a systemic approach rather than a targeted approach. However, the present invention provides an atraumatic method of increasing permeation of tissue by at least one therapeutic drug and/or agent using a pressurized fluid more concentrated with the therapeutic drug and/or agent for a more efficient and uniform distribution of the therapeutic drug and/or agent to the tissue of the targeted location.

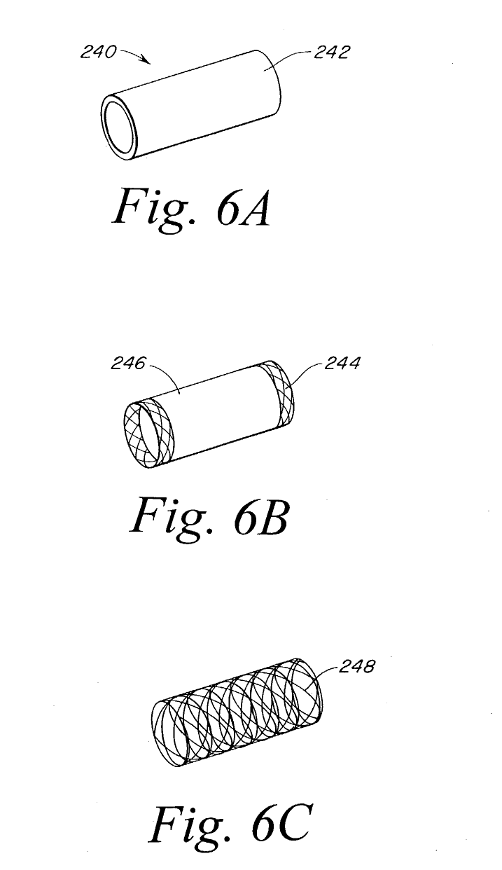

FIGS. 6A, 6B, and 6C illustrate example embodiments of additional medical devices that can be used in conjunction with the expandable device 10. FIG. 6A is a perspective illustration of a stent 240 that is completely encapsulated in a coating 242. FIG. 6B is a perspective illustration of a stent 244 with a partial coating 246. FIG. 6C is a perspective illustration of a stent 248 without a coating, or with a coating on the individual wires of the stent 248. The coating 242 and 246 can be made of PTFE or some other appropriate material as understood by one of ordinary skill in the art. Furthermore, the coating 242 can include one or more therapeutic agents or components for forming therapeutic agents as described herein. The expandable device 10 can be placed within either of the stents 240, 246, or 248 to expand the stents 240, 246, and 248 against a lumen wall within a patient as understood by one of ordinary skill in the art.

In an alternative arrangement, the expandable device 10 can expand within a previously expanded stent (such as stents 240, 246, and 248 of FIGS. 6A, 6B, and 6C). In such an arrangement, the stent 240, 246, or 248 will have already stretched the body lumen or cavity, likely to about 110% of its original inner diameter. The expandable device 10 then expands to meet and compress against the sent 240, 246, or 248 and body lumen internal wall 232. Because the stent 240, 246, or 248 adds additional structure, and the body tissue has already stretched, there is greater force pushing back on the expandable device 10, slightly compressing the expandable device 10 more than in the previously described embodiment. In addition, an increased pressure can be achieved in the expandable device 10 up to about 6 atmospheres, versus the 3 to 4 atmospheres in arrangements without stents 240, 246, or 248.

As previously mentioned, the size and dimensions of the expandable device 10 are determined such that the expandable device 10 can expand to a sufficient diameter relative to the size of an application specific body lumen to create the semi-confined space 234. In other words, if the expandable device 10 is too small, the small orifice 236 will be too large to maintain fluid pressure, and there will be no KIP effect. If the expandable device 10 is too large, the expansion of the expandable device 10 can cause a rupture of the body lumen with application of a substantial pressure. Again, there will be no small orifice 236 unless there is pressurized fluid in the semi-confined space forcing its way out by creating the small orifice 236 with the slight compression of both the body lumen wall and the expandable device 10. The distance between the body lumen and the expandable device 10 (i.e., the height of the orifice) can range between about one ten-thousandth of an inch to about 2 mm. This distance between the body lumen and the expandable device 10 enables the atraumatic delivery of the therapeutic agent and/or drug to the targeted location. With the present invention, there is no highly pressurized jet of fluid ablating the tissue to increase permeation, nor is there a hard structure pressed against the tissue causing tissue damage. The distance between the body lumen and the expandable device 10, caused by the pressurized fluid, protects the tissue from damage.

It has unexpectedly been determined that this pressurized fluid allows the therapeutic agents to preferentially distribute and penetrate into the internal wall 232, which results in a more efficient application of therapeutic drugs or agents into both the intra-cellular and extra-cellular space of the internal wall 232. The resulting therapeutic drug delivery effect is the KIP effect. One result from the more efficient application of the therapeutic drugs or agents is that the dwell time required for application of a specified dosage of therapeutic agent or drug to the targeted location is reduced relative to the previously referenced conventional methods. In addition, if the dwell time is maintained and not reduced, an increased amount of drug or agent permeates the tissue of the targeted location, thus having an improved therapeutic effect relative to prior methods.

Another result is that any fluid containing any therapeutic drugs or agents that do not permeate into the permeation region 238 of the internal wall 232 exits out from the semi-confined space 234 and the fluid pressure decreases to the ambient pressure within the body lumen 230, thereby having no localized drug delivery effect beyond where the KIP effect is applied.

In addition, in arrangements involving a stent 240, 246, or 248 in combination with the expandable device 10, as mentioned previously, a relatively higher pressure is obtained within the expandable device 10 (e.g., up to about 6 atmospheres). The increased pressure results in even further enhancement of therapeutic agent distribution and permeation into the tissue of the body lumen or cavity.

Therapeutic agents applied to the targeted location of the internal wall 232 over time permeate the tissue of the internal wall 232. As described, fluid containing therapeutic agents that do not permeate the internal wall 232 exits the semi-confined space 234 and is diluted and flushed away into the general systemic blood circulation. The fluid applied to the targeted location using the KIP effect can be relatively concentrated with therapeutic agent or drug, with a smaller dosemetric or overall volumetric amount, because of the ability to expose the targeted location to a stream of fluid containing the therapeutic drug and/or agent over a period of time. Therefore, therapeutic agents that do not permeate the body tissue can escape to other portions of the patient's body without ill effect, because of the substantially diluted state of the fluid delivering the agents.

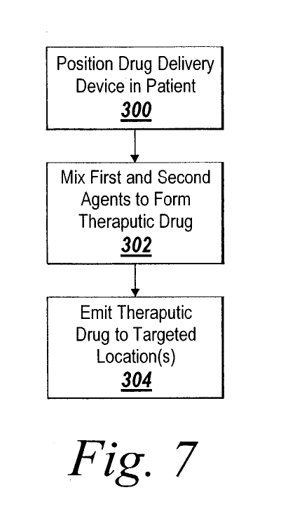

FIG. 7 illustrates one example method for applying a therapeutic drug in accordance with the present invention. The method includes positioning a drug delivery structure, such as the expandable device 10, within a patient's body at a targeted location such as the body lumen 230 (step 300). A first agent or component containing an agent is introduced to the drug delivery structure to react with a second agent or component containing an agent that is disposed within the delivery structure to form the therapeutic drug (step 302). The therapeutic drug then emits from a plurality of locations along the drug delivery structure to the targeted location within the patient at a controlled rate (step 304). If the expandable device 10 is sufficiently sized, and the pressure provided to the expandable device is appropriate, the therapeutic drug can emit using the KIP effect for improved distribution to the tissue and permeation in a reduced dwell time.

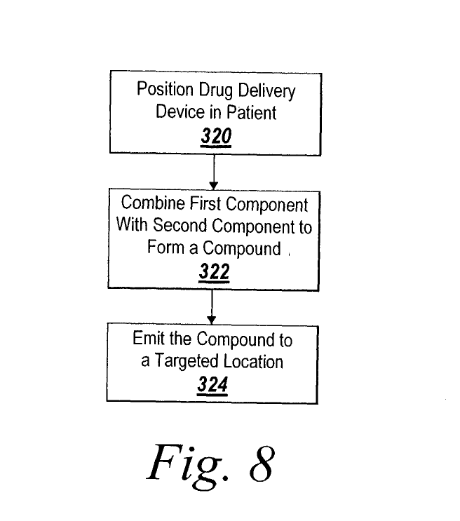

FIG. 8 illustrates an example embodiment of forming a polymeric body within a patient. The method includes positioning a delivery structure, such as the expandable device 10, within the patient at the targeted location (step 320). A first component is introduced to the delivery structure to react with a second component disposed within the delivery structure to form a compound (step 322). The compound emits from a plurality of locations along the delivery structure at a predetermined controlled rate for application to a targeted location to form the polymeric body (step 324). If the expandable device 10 is sufficiently sized, and the pressure provided to the expandable device is appropriate, the therapeutic drug can emit using the KIP effect for improved distribution to the tissue and permeation in a reduced dwell time.

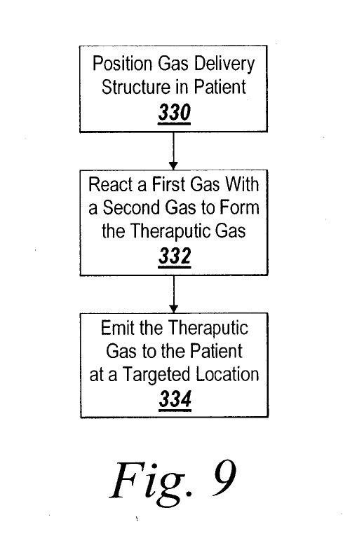

FIG. 9 illustrates an example embodiment of applying a therapeutic gas to a targeted location within a patient's body. A gas delivery structure, such as the expandable device 10, is positioned at the targeted location (step 330). The gas delivery structure receives a first gas to react with a second gas disposed within the delivery structure to form the therapeutic gas (step 332). The therapeutic case is emitted from a plurality of locations along the gas delivery structure at a predetermined controlled rate for application to the targeted location (step 334). If the expandable device 10 is sufficiently sized, and the pressure provided to the expandable device is appropriate, the therapeutic drug can emit using the KIP effect for improved tissue permeation in a reduced dwell time.

In each of the embodiments illustrated in FIGS. 7, 8, and 9, methods discuss a second gas or component being disposed within the delivery structure. It should be noted that the gas or component can exist in the delivery structure in a number of different ways. For example, the second gas or component can be supplied to the delivery structure just prior to, or coincident with, the introduction of the first gas or component to the delivery structure. Alternatively, the second gas or component can be sealed within the delivery structure prior to use by the clinical user. In still another alternative, the component or gas can be resident within the delivery device structure, such as being incorporated into, e.g., PTFE material or other delivery device material, or applied as a coating to the walls of the delivery device structure.

The present invention KIP effect provides for the atraumatic delivery of at least one therapeutic drug and/or agent contained within a pressurized fluid in a substantially uniform drug or agent concentration. More specifically, the present invention KIP effect provides an atraumatic method of increasing permeation of tissue by at least one therapeutic drug and/or agent using a pressurized fluid more concentrated with the therapeutic drug and/or agent for a more efficient and uniform distribution of the therapeutic drug and/or agent to the tissue of the targeted location relative to prior methods. Because of the more efficient drug or agent distribution, the dwell time required for application of a specified dosage of therapeutic agent or drug to the targeted location is reduced relative to prior methods for delivery of a specified dosage of drug or agent. In addition, any fluid containing any therapeutic drugs or agents that do not permeate the body tissue exits out from the semi-confined space. Upon exit, the fluid pressure decreases to the ambient pressure within the body lumen, the drug or agent fluid concentration is diluted and washed away. Therefore, there is no localized drug delivery effect beyond where the KIP effect is applied.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the disclosed invention is reserved.

What is claimed is:

1. A method of delivering a therapeutic agent to a targeted location within a body cavity, comprising:
providing a non-perforated delivery device having at least one wall through which a fluid at first fluid pressure can pass through;
positioning the non-perforated delivery device to provide a radial fluid force against the targeted location;
supplying the fluid including at least one therapeutic agent to the therapeutic agent delivery device at the first fluid pressure;
the fluid passing through the at least one wall of the delivery device to create a semi-confined space external to the delivery device at a second fluid pressure; and
the delivery device applying the radial fluid force against the semi-confined space and the fluid disposed therein while simultaneously facilitating the fluid passing through the delivery device to maintain the second fluid pressure in the semi-confined space at the targeted location;
wherein the fluid contains at least one therapeutic agent that is distributed to the targeted location in a substantially uniform distribution in an amount sufficient to create a therapeutic effect modulatable by the fluid pressure and a dwell time.

2. The method of claim 1, wherein the semi-confined space comprises a chamber formed by the targeted location and an external wall of the delivery device, and having an orifice along a perimeter of the therapeutic agent delivery device through which the fluid can flow.

3. The method of claim 2, wherein the orifice forms upon introduction of the fluid, under pressure, external to the delivery device.

4. The method of claim 1, wherein the first fluid pressure is greater than the second fluid pressure.

5. The method of claim 1, wherein the second fluid pressure is greater than an ambient pressure external to the delivery device and the semi-confined space.

6. The method of claim 1, further comprising supplying the fluid to the delivery device using a catheter coupled with the delivery device.

7. The method of claim 1, wherein the at least one wall is collapsible and expandable.

8. The method of claim 7, wherein the delivery device applying the radial fluid force against the targeted location comprises introducing the fluid to the delivery device at the first fluid pressure to expand the delivery device to an increased effective diameter, resulting in the application of the radial fluid force.

9. The method of claim 1, wherein the at least one wall is fixed in shape.

10. The method of claim 9, wherein the delivery device applying the radial fluid force against the targeted location comprises implanting the delivery device in the body cavity, the delivery device having an effective diameter greater than an effective diameter of the body cavity.

11. The method of claim 1, further comprising the radial fluid force expanding the body cavity to between about 101% and about 150% of a pre-implantation body cavity effective diameter.

12. The method of claim 1, wherein the delivery device comprises an irrigating shaped form.

13. The method of claim 1, further comprising adjusting the dwell time to modulate an amount of therapeutic agent delivered to the targeted location.

14. The method of claim 1, further comprising modulating at least one of the fluid pressure, a concentration of the therapeutic agent in the fluid, and the dwell time to modulate an amount of therapeutic agent delivered to the targeted location.

15. A therapeutic agent delivery device suitable for positioning at a targeted location within a body cavity, comprising:
a non-perforated wall structure having a porosity enabling a fluid to pass through at a first fluid pressure, the fluid including at least one therapeutic agent; and
at least one supply aperture formed in the wall structure providing access for supplying the fluid to the therapeutic agent delivery device;
wherein the wall structure is sized to generate a radial fluid force against the targeted location upon implantation to enable creation of a semi-confined space using the fluid at a second fluid pressure; and
wherein the wall structure applies the radial fluid force against the targeted location while simultaneously facilitating the fluid passing through the wall structure to maintain the second fluid pressure in the semi-confined space external to the wall structure at the targeted location, such that the therapeutic agent contained within the fluid is substantially uniformly distributed to the targeted location in a substantially in an amount sufficient to create a therapeutic effect modulatable by the fluid pressure and a dwell time.

16. The therapeutic agent delivery device of claim 15, wherein the semi-confined space comprises a chamber formed by an the targeted location and an external side of the wall structure, and having an orifice along a perimeter of the therapeutic agent delivery device through which the fluid can flow.

17. The therapeutic agent delivery device of claim 16, wherein the orifice forms upon introduction of the fluid, under pressure, external to the wall structure.

18. The therapeutic agent delivery device of claim 15, wherein the first fluid pressure is greater than the second fluid pressure.

19. The therapeutic agent delivery device of claim 15, wherein the second fluid pressure is greater than an ambient pressure external to the therapeutic agent delivery device and the semi-confined space.

20. The therapeutic agent delivery device of claim 15, wherein access for supplying the fluid to the therapeutic agent delivery device comprises a catheter coupled with the at least one supply aperture.

21. The therapeutic agent delivery device of claim 15, wherein the wall structure is collapsible and expandable.

22. The therapeutic agent delivery device of claim 21, wherein the radial fluid force against the targeted location results from introduction of the fluid to the therapeutic agent delivery device at the first fluid pressure.

23. The therapeutic agent delivery device of claim 15, wherein the wall structure is fixed in shape.

24. The therapeutic agent delivery device of claim 23, wherein the radial fluid force against the targeted location results from implantation of the therapeutic agent delivery device in the body cavity.

25. The therapeutic agent delivery device of claim 15, wherein the radial fluid force expands the body cavity to between about 101% and about 150% of a pre-implantation body cavity effective diameter.

26. The therapeutic agent delivery device of claim 15, wherein the wall structure comprises an irrigating shaped form.

27. The method of claim 15, further comprising adjusting the dwell time to modulate an amount of therapeutic agent delivered to the targeted location.

28. The method of claim 15, further comprising modulating at least one of the fluid pressure, a concentration of the therapeutic agent in the fluid, and the dwell time to modulate an amount of therapeutic agent delivered to the targeted location.
